(19) United States
(12) Patent Application Publication
(10) Pub. No.: US 2008/0272918 A1
(43) Pub. Date: Nov. 6, 2008

(54) MEDICAL DEVICE AND METHOD FOR PREVENTING FALLS

(76) Inventor: SALLY INGERSOLL, Des Moines, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 11/744,545

(22) Filed: May 4, 2007

(51) Int. Cl. G08B 23/00; A61B 5/12

(52) U.S. Cl. 340/573.1; 340/665; 340/686.1; 600/595

(57) ABSTRACT

A medical device is beneficial to protect a patient in a care facility from a fall by remotely alerting a caregiver of patient's unsafe mobility before the fall using a motion sensor having an alarm. The medical device uses an operating module with a wireless transmitter integrated into the motion sensor for broadcasting the alarm as an alert signal to notify the caregiver of patient's unsafe motion. A wireless receiver with the caregiver receives the alert signal from the wireless transmitter. The alert signal identifies the motion sensor that was activated so the caregiver can respond to prevent the patient from falling.

### FIELD OF THE INVENTION

The present invention relates to a medical device and method for preventing falls to prevent at-risk patients by monitoring for unsafe attempts at mobility. More particularly, the present invention relates to a medical device and method for integration into a mobility sensor, such as a pressure sensor in the chair or bed in which the patient sits or lies, that alerts a caregiver of the patients movement by converting the alarm into an alert signal broadcast from the mobility sensor by way of a wireless transmitter to a wireless signal receiving device with the caregiver. The present invention omits the need for current, audible alert systems.

### BACKGROUND OF THE INVENTION

Some nursing home residents have limited mobility with a risk of injury from falling. It is common in such care facilities to use a sensor with such patients to sense when they move unassisted from a bed or chair and to notify the nurses or caregivers that immediate attention is needed to assist the patient.

Many existing patient motion sensors when activated produce loud, disturbing, audible alarms to alert caregivers of the location of the tripped sensor and the patient in need of care. These audible alarms disturb, annoy and often confuse patients. Therefore, there is a need for a medical device and method that modifies audible alarm sensors by incorporating a wireless signal transmitter into the sensor for transmitting an inaudible alert signal in place of the audible alert tone to notify a caregiver of the need to respond.

Motion detectors for monitoring patient movement are known in the prior art. For example, U.S. Pat. No. 7,071,820 to Callaway discloses a patient ambulation motion detector worn by the patient. Motion detectors that are positioned on, attached to, or worn by the patient do not solve the problem of patient motion in many circumstances. For example, patients suffering from illnesses, such as dementia, or those in a Chronic Confusion or a Dementing Illness (CCDI) unit, are often highly resistive to any devices worn on or attached to their body.

Many care facilities currently have installed and use pressure and fixed position motion sensors to detect patient's mobility in order to prevent patients from falling. If tripped, these sensors produce a loud alarm to notify the caregiver. In some instances, pressure sensors are positioned in a bed, chair or wheelchair beneath the patient. Sensors are also often positioned where a patient would step in an attempt to get up from a bed or chair. In some cases, motion sensors are placed in areas adjacent a bed, chair or doorway to detect movement from the chair, bedside or doorway. In other applications, such as where the patient attempts to release themselves from a merry-walker, a magnetic alarm alerts a caregiver if the patient is too far from the safety seat. Many care facilities have installed and currently use many of the aforementioned sensors. Therefore, there is a need for a medical device and method that can be easily integrated into an motion sensor to replace the startling, loud, audible alarm with an inaudible alert signal transmitted to a wireless receiver with the caregiver.

In care facilities, such as a CCDI unit, audible alarms can be heard going off sometimes several at a time. This means that the caregivers must quickly distinguish between the alarms to determine the location of each alarm and patient in need. The process of responding to concurrently tripped alarms can be confusing and frustrating, resulting in a longer response time and imprudent use of resources. Therefore, there is a need for a medical device and method having a wireless receiver that processes signals from multiple tripped alarms and displays or alerts which sensor has been activated, the patient, patient number, room number, and/or patient location. To solve this same problem, there is a further need to provide a medical device and method wherein the wireless receiver has a cancel call and/or a reset button so the first responder can cancel the alert call, reset the sensor, or permit the sensor to continue transmitting an alert signal if further assistance is needed.

### BRIEF SUMMARY OF THE INVENTION

Therefore, it is an object, feature, or advantage of the present invention to improve over the state of the art.

It is a further object, feature, or advantage of the present invention to provide a medical device and method replacing loud, disturbing, audible alarm motion sensors with a silent alert signal broadcast directly to the caregiver.

Yet another object, feature, or advantage of the present invention is to provide a medical device and method for preventing falls by alerting the caregiver of a patient's movement before the fall.

A further object, feature, or advantage of the present invention is to provide a medical device and method for retrofitting patient motion sensors, such as motion and pressure sensors, with an operating module and a transmitter for communicating an alert signal directly to a wireless receiver.

Yet another object, feature, or advantage of the present invention is to provide a medical device and method wherein the caregiver can cancel and/or reset the alert signal from a triggered sensor using a wireless receiver.

A still further object, feature, or advantage or the present invention is to provide a medical device and method for detecting patient movement by patients resistive to wearing or having an motion detection device attached to them.

One or more of these and/or other objects, features, or advantages of the present invention will become apparent from the specification and claims that follow.

According to one aspect of the present invention a medical device to protect a patient in a care facility from a fall by remotely alerting a caregiver of patient mobility before the fall is disclosed. The medical device has a mobility sensor, an operating module and a wireless transmitter integrated into the sensor for broadcasting as an alert signal to notify the caregiver of at-risk motion by a particular patient. The medical device also has a wireless receiver with the caregiver for receiving the alert signal from the wireless transmitter. The alert signal identifies the motion sensor that was activated so the caregiver can respond to prevent the patient from falling. In the preferred form, the wireless receiver has a reset button so the caregiver who responds first can send a reset signal from the wireless receiver to the motion sensor, the motion sensor is a pressure sensor positioned beneath where the patient lies or sits, and the alert signal from the wireless transmitter displays on the wireless receiver the motion sensor activated, patient name, patient number, room number, and/or patient location, and the wireless receiver sends a programming signal to the motion sensor to thereby program the operating module.

A new method for protecting a patient from falling by remotely alerting a caregiver of patient mobility within a care facility is also disclosed. The method includes placing an motion sensor and a wireless transmitter in a patient's room and giving a wireless receiver to a caregiver, the wireless receiver having a reset button and a cancel call button. The method also includes integrating an operating module together with the wireless transmitter into the motion sensor, broadcasting an alert signal when the motion sensor is activated to the wireless receiver to alert the caregiver of patient movement, and sending a reset or a cancel call signal from the wireless receiver to the operating module using the rest or the cancel call button on the wireless receiver. In the preferred form, the method includes the steps of programming the wireless transmitter using the wireless receiver to identify the patient, sensor location, room number, and/or patient number, converting an alarm from the motion sensor to the alert signal for inaudible wireless broadcasting of the alert signal to prevent the patient from becoming annoyed, scared, or excited, displaying on the wireless receiver the patient, sensor location, room number, patient location, and/or patient number, activating the pre-existing sensor using the wireless receiver, and configuring an antenna within the care facility to relay the alert signal between the wireless transmitter and the wireless receiver.

A system comprising a medical device for protecting a patient from falling by remotely alerting a caregiver of patient motion before the fall using a motion sensor with an alarm is disclosed. The system comprises a care facility using the motion sensor with the alarm, a wireless transmitter connected to an operating module integrated into the motion sensor to replace the alarm to a silent alert signal broadcast within the care facility, and a wireless receiver worn by the caregiver for receiving and displaying the alert signal from the wireless transmitter to thereby identify the motion sensor that was activated so the caregiver can respond to prevent the patient from falling.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a pictorial representation of the sensors and notification process of the prior art.

FIG. 2 is a pictorial representation illustrating an exemplary embodiment of the medical device and method for remotely alerting a caregiver when the sensor is activated.

FIG. 3A is a perspective view of an exemplary embodiment of the wireless receiver.

FIG. 3B is a perspective view of another exemplary embodiment of the wireless receiver.

FIG. 3C is a perspective view of an additional exemplary embodiment of the wireless receiver.

FIG. 3D is an enlarged view of the display for the wireless receiver in FIG. 3C.

FIG. 4A is a flow diagram illustrating an exemplary embodiment of the present invention.

FIG. 4B is a continuation of the flow diagram of FIG. 4A.

### DETAILED DESCRIPTION OF THE PREFERRED EMBODIMENT

The present invention includes a number of aspects, all of which have broad and far-reaching application. One aspect of the present invention relates to replacing loud, disturbing, audible alarms or optionally a silent alarm from a motion sensor with an inaudible signal broadcast directly to a caregiver. Another aspect of the present invention prevents a patient from falling by utilizing a motion sensor alerting the caregiver of the patient's movement before the fall. Another aspect of the present invention relates to a medical device and method for retrofitting a motion sensor, such as a motion and/or pressure sensor, with an operating module and a transmitter for communicating an alert signal directly to a wireless receiver. Still another aspect of the present invention relates to a medical device and method wherein a caregiver can cancel and/or reset the alert signal from the triggered sensor using the wireless receiver. Although specific embodiments are described herein, the present invention is not to be limited to these specific embodiments. The present invention contemplates numerous other options in the design and use of the medical device and method.

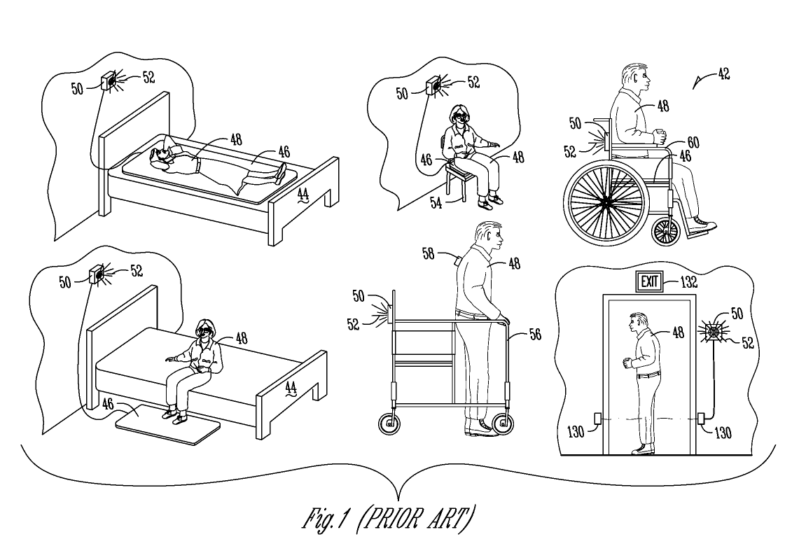

FIG. 1 is illustrative of the prior art. FIG. 1 shows several different patient motion sensors and the method they employ to alert a caregiver of the need to respond to assist the patient 48. Hospitals, nursing homes and similar or related care facilities use motion sensors to detect patient movement. These sensors can be a pressure sensor pad positioned beneath the patient 48 where the patient sits or lies. If activated, these sensors use an audible alarm to alert a caregiver of the need for response. For example, if patient 48 attempts to get up from the bed 44, the motion sensor 50 is triggered which produces an audible alarm 52 in the patient's room for alerting the caregiver. A caregiver then must detect the room where the audible alarm 52 originated to render assistance to the patient 48. Another similar example and one commonly used entails positioning a pressure pad 46 on the floor near the side of the bed 44 of the patient 48. The pressure pad 46 is wired to the motion sensor 50. In this instance, if the patient 48 attempts to put his or her feet down from the bed 44, the pressure pad 46 is activated causing the motion sensor 50 to produce an audible alarm 52 in the patient's room to thereby alert the caregiver of the patient's movement. In another application, the patient 48 may be seated in a chair 54. A pressure pad 46 is positioned on the chair beneath the patient 48 for detecting patient movement. Again, the pressure pad 46 is wired to the motion sensor 50 which produces an audible alarm 52 for alerting the caregiver if the patient 48 attempts to move from the chair 54. Similar to the patient 48 sitting in the chair 54, a pressure pad 46 may be positioned beneath the patient 48 sitting in a wheelchair 60 to alert the caregiver if the patient 48 attempts to get up from the wheelchair 60. Another device needing monitored is a merywalker 56, which allows the patient 48 freedom of movement. An motion sensor 50 with an audible alarm 52 is configured to emit an audible alarm 52 when a magnetic sensor 58 on the backside of the patient 48 moves outside of the some safe distance indicative of the patient 48 trying to remove him or herself from the merywalker 56.

Another prior art sensor used to detect patient mobility is a motion sensor 130. Motion sensor 130 is positioned at points near the bed 44, chair 54 and/or door/exit 132. For example, one type of motion sensor 130 used to detect patient movement is like the one shown in FIG. 1. Here patient motion is detected when the beam between two components of the motion sensor 130 is broken. For instance, if the patient 48 attempts to proceed through the exit 132, the motion sensor 130 is activated and the motion sensor 50 emits an audible alarm 52 to alert the caregiver.

In all of the instances of patient motion monitoring shown in FIG. 1, the caregiver only is made aware of the patient's movement and/or subsequent need for assistance by the audible alarm 52 in the patient's room, which notifies the caregiver of the need for response. For the caregiver to respond promptly, the caregiver must be able to quickly determine from where the audible alarm 52 is coming. To the extent the caregiver is not able to quickly and accurately determine a location of the audible alarm 52, the patient 48 may suffer some consequence. For example, the caregiver may arrive too late to keep the patient 48 from falling from the wheelchair 60, or bed 44, and/or chair 54. Additionally, the audible alarm 52, by necessity, is often a loud, disturbing and frightening noise that can frighten, annoy, scare and/or alarm the patient 48. Such alarms are inherently dangerous operating in an environment where the patient 48 could be injured as a result of his or her response or quick movement upon hearing the audible alarm 52. One example of an assisted living setting where this danger is particularly relevant is a chronic confusion or dementing illness unit, or a unit where the patients suffer from dementia. In addition to surprising or frightening the patient, an audible alarm 52 is naturally disruptive and may interrupt the natural sleep habits of each patient 48. These loud alarms can also disrupt patients in other rooms.

Another concern of audible alarms 52 is that more than one may be triggered at the same time. This makes the process very challenging for distinguishing between audible alarms 52, to determine the location of the patient 48. This ultimately keeps the patient 48 from receiving timely care and is a waste of the care facility's resources. Still, even if the caregiver is able to quickly distinguish between multiple audible alarms 52, the remaining triggered audible alarms 52 will continue to emit a loud alarm until a caregiver is able to respond and deactivate the audible alarm 52. Thus, in instances where several audible alarms 52 have been activated simultaneously, these alarms 52 continue to disturb, annoy and frighten one or more patients until the caregiver can respond to each patient 48 and deactivate the audible alarm 52. Thus, as indicated from the foregoing description, the prior art used to alert a caregiver of patient unsafe motion is highly inefficient, disruptive to patient living and unsafe.

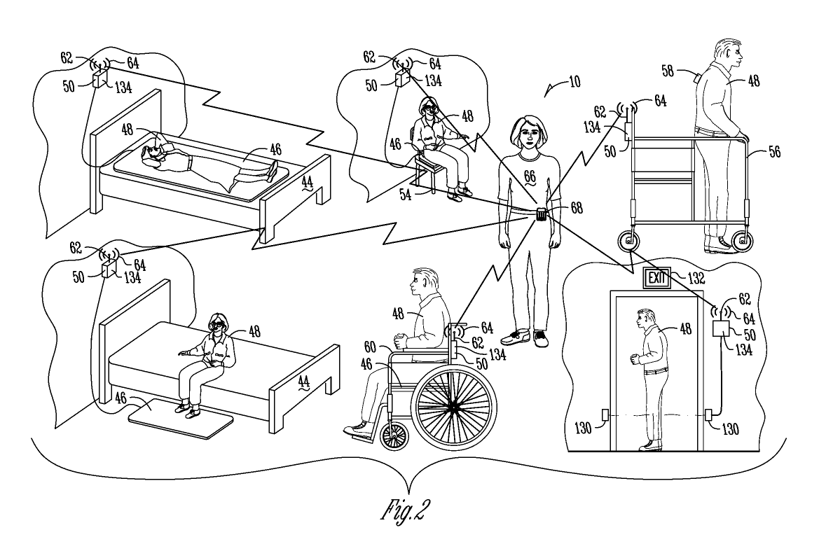

Illustrated in FIG. 2 is an exemplary embodiment of the present invention which overcomes all of the disadvantages of the prior art shown in FIG. 1. In FIG. 2, a caregiver 66 is shown having a wireless receiver 68. The wireless receiver 68 can be any type of receiving unit capable of wirelessly receiving some signal. Thus, in FIG. 2, the wireless receiver 68 is configured to receive alert signals 54 emitted from wireless transmitter 62. Similarly, wireless receiver 68 could also be configured to receive an alert signal 64 from a relay point or antenna designed to amplify the signal to ensure the wireless receiver 68 receives the alert signal 64 regardless of the caregiver's 66 position relative to the wireless transmitter 62.

As shown in FIG. 2, the present invention uses the motion sensors 50 to monitor patient movement without an audible alarm. For example, in one embodiment, patient 48 rests atop of pressure pad 46 on the bed 44. The pressure pad 46 is in signal communication with motion sensor 50. Motion sensor 50 is integrated with an operating module 134 and a wireless transmitter 62 that emits an alert signal 64. This alert signal 64 is received at the wireless receiver 68 worn by the caregiver 66. Thus, in this instance, if the patient 48 attempts to get up from the bed 44, the pressure pad 46 triggers the motion sensor 50 having an operating module 134 which generates the alert signal 64 that is emitted from the wireless transmitter 62 to the wireless receiver 68 to alert the caregiver 66 of the patient's movement. The motion sensor 50, wireless transmitter 62, alert signal 64, and wireless receiver 68 operate in the same manner as previously discussed for all other instances of patient unsafe motion shown in FIG. 2. For example, if the patient 48 attempts to get up from the wheelchair 60, the pressure pad 46 detects the movement of patient 48 by way of motion sensor 50. The operating module 134 processes the data and sends a wireless alert signal 64 by way of wireless transmitter 62 to the wireless receiver 68 worn by the caregiver 66. In this manner, the caregiver 66 is alerted of the patient's movement and the need to respond. The wireless alert signal 64 sent from the wireless transmitter 62 to the wireless receiver 68 is inaudible. Thus, the alert signal 64 will not frighten, scare, or annoy or possibly cause the patient 48 to make an unsafe decision because the patient 48 is unaware of the alert signal 64 being transmitted. The receiver 68 generates an audible sound and/or vibration to alert or notify the caregiver 66 that the patient has moved beyond an acceptable range.

To relay the alert signal 64 to the caregiver 66, a wireless transmitter 62 and operating module 134 is integrated or configured into the motion sensor 50. Because the wireless transmitter 62 and operating module 134 can be integrated or configured into the motion sensor 50, the present medical device represents a significant cost savings over other patient motion sensors which require altogether new sensors to be purchased, installed and networked throughout the care facility. Furthermore, the wireless transmitter 62 and operating module 134 could be adapted to operate using the preexisting power source associated with the motion sensor 50 or replace their signal device, such as where the motion sensor 50 is powered by an electrical outlet. The wireless transmitter 62 and operating module 134 could be further adapted and configured to operate functions, features and/or controls associated with the motion sensor 50 and/or sensor 46, 130 and 58.

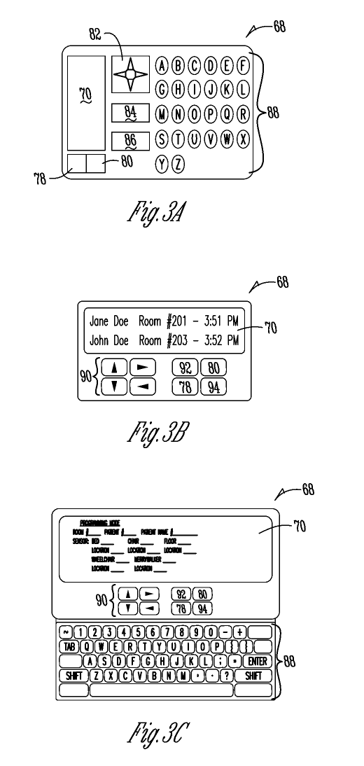

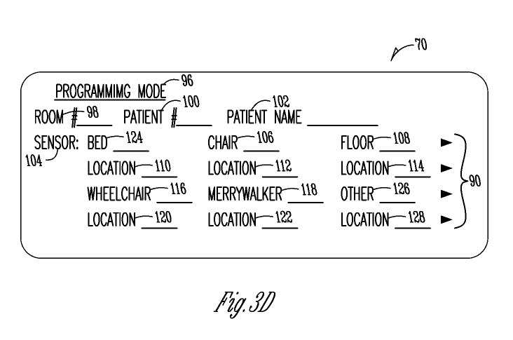

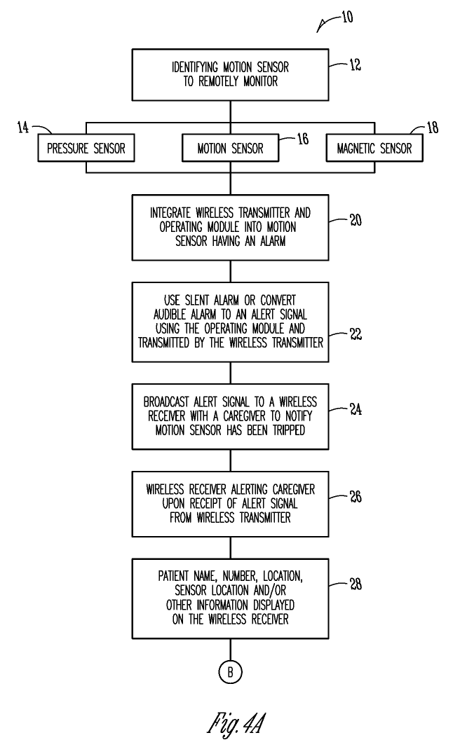

Another advantage of the present invention is illustrated in FIGS. 3A-D. FIGS. 3A-D are illustrative of a few exemplary embodiments of the wireless receiver 68. The wireless receiver 68 is preferably a device that can be worn or carried by the caregiver 66 (shown in FIG. 2). However, the wireless receiver 68 could be a stationary unit in addition to the one worn by the caregiver 66. The stationary unit could be positioned at a desk or central administrative station within a care facility. For example, the wireless receiver 68 could be positioned and powered by an electrical outlet at a nurses station. In the preferred embodiment, the wireless receiver 68 would be configured or adapted to convey information to the caregiver 66 to alert the caregiver 66 of the motion sensor 50 that has been activated as well as any other relevant information to help the caregiver respond quickly, efficiently, and responsibly to the needs of each patient 48. For example, one exemplary embodiment of the wireless receiver 68 may be a pager-like device as shown in FIG. 3B. This wireless receiver 68 has a display 70, scroll buttons 90, a cancel call button 92, a reset button 78, an activate/deactivate button 80 and an on/off button 94. The wireless receiver 68 of FIG. 3B is configured to communicate with the wireless transmitter 62 (shown in FIG. 2) using hardware associated with such devices and with techniques that are well within the knowledge of those skilled in this art.

The wireless receiver 68 of FIG. 3B could be further configured to display information on the display 70 about the sensor that has been triggered. The display 70 may be a computer monitor or other screen operatively connected to a data processor. For example, this could be done by displaying the name of the patient, room number and/or time that the sensor was triggered. Additionally, if multiple sensors are triggered simultaneously, or in a relatively short period of time, the display 70 could show the order in which the sensors are triggered by illustrating the time in which each sensor was triggered. The information presented on the display 70 would allow the caregiver (shown in FIG. 2) to quickly respond to the specific location where the sensor was triggered and/or the patient's location. Scroll buttons 90 would allow the caregiver to scroll through the information shown on the display 70. Other buttons on the wireless receiver 68 could allow the caregiver 66 to independently control each motion sensor 50 by sending a signal from the wireless receiver 68 to the wireless transmitter 62. Thus, for example, the caregiver 66 could use the cancel call button 92 to cancel the alert signal being emitted from the wireless transmitter 62. The ability of the caregiver 66 to cancel the call using the wireless receiver 68 has many benefits. For example, if the caregiver can visually see that the patient 48 is okay, but cannot yet respond, the caregiver 66 can cancel the call and respond when able. Furthermore, having the wireless receiver 68 with the caregiver 66 allows the caregiver 66 to cancel a call without having to do it at the motion sensor 50. If the patient 48 has fallen, the caregiver 66 can render assistance to the patient 48 and reset the call button on the motion sensor 50 without having to leave the patient's side. The wireless receiver 68 could also be configured so that if the caregiver 66 cancels the call, the other wireless receivers are automatically updated with the new information so that more than one caregiver 66 is not responding to each call. Alternatively, the caregiver 66 could continue to allow the motion sensor 50 to emit alert signal 64 so that additional caregivers 66 respond if further assistance is needed. Additionally, caregiver 66 could use wireless receiver 68 to notify other caregivers 66 of the need for additional help. Another possible feature of the wireless receiver 68 is a reset button 78. The reset button 78 would allow the caregiver 66 to reset the motion sensor 50. For instance, where the caregiver 66 has responded and helped patient 48 back onto the bed 44, the caregiver 66 can reset the pressure sensor 46 using the reset button 78 on the wireless receiver 68.

It may also be desirable to activate and/or deactivate one or more sensors using the wireless receiver 68. Thus, an activate/deactivate button 80 is configured into the wireless receiver 68 so that the caregiver 66 can activate or deactivate one or more sensors as desired. For example, if the patient's health is such that he or she can get up from the bed 44 and move around without fear of harm or accident, the caregiver 66 could deactivate the pressure pad 46 using the wireless receiver 68. An on and off switch 94 could be used to toggle the wireless receiver 68 from an on state to an off state, as is customary with electronic devices.

FIG. 3A shows another embodiment of the wireless receiver 68. The wireless receiver 68 in FIG. 3A has a display 70, reset button 78, activate/deactivate button 80 and a keyboard 88. The wireless receiver 68 also has a compass locator 82 and an activate/deactivate locator button 84, as well as a reset locator button 86. Compass locator 82 could be used to track and monitor the location of the patient 48 in such cases where the patient is mobile. For example, if the patient is in a merywalker 56 or wheelchair 60, the wireless receiver 68 could be adapted and configured to display the origin of the alert signal 64 on compass locator 82. The activate/deactivate locator button 84 would allow the caregiver 66 to activate/deactivate the locator 82 feature. Similarly, the caregiver 66 could use the reset locator button 86 to reset the locator 82 feature.

FIG. 3C illustrates another embodiment of the wireless receiver 68 similar to the wireless receiver 68 shown in FIG. 3B but having a keyboard 88. The keyboard 88 in FIGS. 3A and 3C would allow the caregiver 66 to enter information into the wireless receiver 68 that could be shared with other wireless receivers 68 and/or communicated to motion sensors 50 by way of wireless transmitter 62. For example, the wireless receiver 68 could have a programming mode 96 as shown in FIG. 3D. In the programming mode 96, the caregiver 66 could use the wireless receiver 68 to program information into the motion sensor 50 using the operating module 134. For example, the display 70 in FIG. 3D shows some of the relevant indicia that caregiver 66 could use to program the operating module 134 of the motion sensor 50. Additionally, the caregiver 66 could use the wireless receiver 68 in the programming mode 96 to coordinate the information that is presented to the caregiver 66 on the display 70. This information could be specific to each motion sensor 50 that is triggered. For example, the caregiver 66 might enter the room number 98, patient number 100 and/or patient name 102 associated with each motion sensor 50. The caregiver 66 could distinguish between each motion sensor 50 by indicating which sensor 104 is to be monitored. The sensor 104 could be associated with the specific room number 98, patient number 100 and/or patient name 102. If there are several different motion sensors 50 positioned within the patient's room, the caregiver 66 could program the operating module 134 of each motion sensor 50 using programming mode 96 to identify each of the individual types of sensors. One sensor may be positioned on the bed 124, chair 106 and/or floor 108. The caregiver 66 could also describe the location 110 for the bed 124, location 112 for the chair 106, and/or location 114 for the floor 108. Similarly, the caregiver 66 could program the wireless receiver 68 to monitor signals broadcast from any wheelchair 116, merywalker 118 or some similar or other related sensor 126. The location 120 for the wheelchair 116, location 122 for the merywalker 118 and location 128 for the other sensor 126 could also be programmed. Scroll buttons 90 on the display indicate to the caregiver 66 that the caregiver 66 can scroll through the information to navigate to other information as is customary with electronic devices. Because the caregiver 66 can program each operating module 134 of each motion sensor 50 using the wireless receiver 68, the caregiver 66 can control the information that is broadcast from each motion sensor 50 when the sensor is triggered.

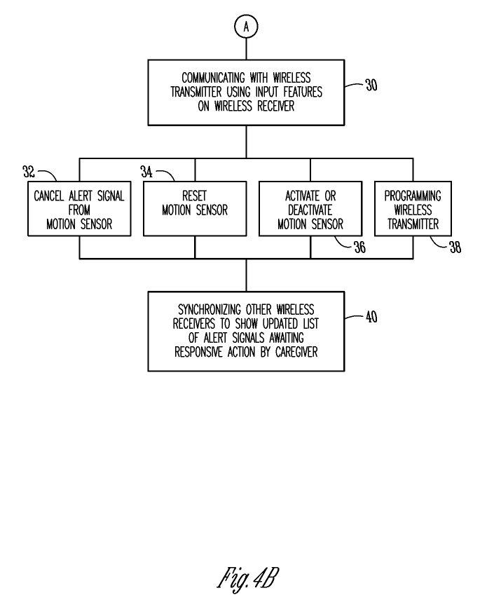

FIGS. 4A and B illustrate a flow diagram according to an exemplary embodiment of the present invention. In particular, the medical device 10 entails identifying the motion sensor 50 to remotely monitor as shown in Step 12. As previously discussed, care facilities have and use a variety of motion sensors to monitor patient movement. These sensors entail but are not limited to pressure sensors 14, motion sensors 16 and/or magnetic sensors 18. Upon identification of the sensor to remotely monitor, a wireless transmitter and operating module is integrated or replaces the signal device of the motion sensor according to Step 20. The pre-existing pressure sensor 14 and motion sensor 16 and/or magnetic sensor 18 used in care facilities often use an alarm to alert the caregiver when the sensor has been activated. In Step 22 the alarm is converted or replaced by an alert signal using the operating module and transmitted by the wireless transmitter. The operating module can be programmed to automatically convert the alarm to an alert signal, and alternatively, may be configured with a switch so that the caregiver can switch between the motion sensor emitting an alarm and/or an alert signal. The operating module may be integrated into the motion sensor so as to communicate with the motion sensor and the wireless transmitter using hardware and components that are within the knowledge of those skilled in the art of electronics and wireless transmission of a signal. The operating module contains all of the necessary electronics and hardware for converting the alarm to an alert signal, transmitting the alert signal, programming and controlling the motion sensor. The operating module communicates with the wireless transmitter to broadcast, as illustrated in Step 24, the alert signal to a wireless receiver worn by or with a caregiver to notify the caregiver that the motion sensor has been activated. In Step 26 the wireless receiver, upon receiving the alert signal from the wireless transmitter, alerts the caregiver that the motion sensor has been activated. The wireless receiver alerts the caregiver by those methods that are well known in the art such as vibration, an alert tone and/or a combination of both. Shown in Step 28, the operating module wirelessly communicates information from the wireless transmitter to the wireless receiver to notify the caregiver of such indicia as the patient's name, patient's room number, patient's location, the sensor location, and/or any other information relevant to rendering assistance to the patient that could be displayed on the wireless receiver.

In FIG. 4B, Step 30 shows that the caregiver can communicate with the operating module within the motion sensor by way of the wireless transmitter using input features on the wireless receiver. For example, the caregiver can cancel the alert signal from the motion sensor (Step 32), reset motion sensor (Step 34), activate or deactivate motion sensor (Step 36) and/or program the motion sensor using the wireless transmitter and operating module (Step 38). The wireless receivers could be synchronized to show an updated list of the alert signals awaiting responsive action as shown in Step 40. For example, if a caregiver responds to a triggered motion sensor, the caregiver can cancel the alert signal from the motion sensor (Step 32), which will update the information being displayed on other wireless receivers. This would allow each caregiver to have an updated status of each motion sensor and/or any action taken whether programming or otherwise to interface with any of the motion sensors.

The preferred embodiment of the present invention has been set forth in the drawings, specifications and those specific terms are employed, either used in the generically description sense only and are not used for the purposes of limitation. Changes in the formed proportion of parts as well as in the substitution of equivalents are contemplated as circumstances may suggest or are rendered expedient without departing from the spirit and scope of the present invention as further defined in the following claims.

What is claimed is:

1. A medical device to protect a patient in a care facility from a fall by remotely alerting a caregiver of patient's motion before the fall comprising:
a motion sensor;
an operating module and a wireless transmitter in communication with the motion sensor for broadcasting an alert signal to notify the caregiver of patient movement by a particular patient;
a wireless receiver with the caregiver for receiving the alert signal from the wireless transmitter; and
the alert signal for identifying the motion sensor that was activated so the caregiver can respond to prevent the patient from falling.

2. The medical device of claim 1 wherein the wireless receiver has a reset button so the caregiver who responds first can send a reset signal from the wireless receiver to the motion sensor.

3. The medical device of claim 1 wherein the motion sensor is a pressure sensor positioned beneath where the patient lies or sits.

4. The medical device of claim 1 wherein the motion sensor is positioned to detect patient movement before the fall.

5. The medical device of claim 1 wherein the alert signal from the wireless transmitter displays on the wireless receiver the motion sensor activated, patient name, patient number, room number, and/or patient location.

6. The medical device of claim 1 wherein the wireless receiver sends a programming signal to the motion sensor to thereby program the operating module.

7. The medical device of claim 1 wherein the wireless receiver is a pager, a handheld or a cellular device.

8. A medical device to protect a patient in a care facility from a fall by remotely alerting a caregiver of patient's motion before the fall comprising:
a motion sensor having a silent alarm;
a wireless transmitter powered by and integrated into the motion sensor;
an operating module adapted to convert the alarm to an alert signal broadcast from the wireless transmitter within the care facility;
a wireless receiver with the caregiver for receiving and displaying the alert signal from the wireless transmitter;
a reset button on the wireless receiver so the caregiver can send a reset signal from the wireless receiver to the operating module to reset the motion sensor; and
the alert signal for identifying the motion sensor that was activated so the caregiver can respond to prevent the patient from falling.

9. The medical device of claim 8 wherein the wireless receiver signals the caregiver with audible tones, visual cues, and/or vibration pulses when the motion sensor is activated.

10. The medical device of claim 8 wherein the wireless receiver is programmed to identify alert signals from multiple motion sensors.

11. The medical device of claim 8 wherein the motion sensor is a pressure sensor positioned to monitor unsafe motion of the patient.

12. The medical device of claim 8 wherein the wireless receiver has a visual display to interface with and program the operating module.

13. The medical device of claim 8 wherein the wireless receiver sends a programming signal to the operating module to cancel calls from, identify location of, activate, deactivate, and/or reset the motion sensor.

14. The medical device of claim 8 wherein the operating module associated with each motion sensor is programmed to broadcast information about the patient, room number, and/or location when the motion sensor is activated.

15. A method for protecting a patient from falling by remotely alerting a caregiver of patient motion within a care facility, the method comprising:
placing a motion sensor and a wireless transmitter in a patient's room;
giving a wireless receiver to a caregiver, the wireless receiver having a reset button and a cancel call button;
integrating an operating module together with the wireless transmitter into the motion sensor;
broadcasting an alert signal when the motion sensor is activated to the wireless receiver to alert the caregiver of patient's unsafe motion; and
sending a reset or a cancel call signal from the wireless receiver to the operating module using the rest or the cancel call button on the wireless receiver.

16. The method of claim 15 further comprising the step of programming the operating module using the wireless receiver to identify the patient, sensor location, room number, and/or patient number.

17. The method of claim 15 further comprising the step of converting an alarm from the motion sensor to send the alert signal for inaudible wireless broadcasting of the alert signal to prevent the patient from becoming annoyed, scared, or excited.

18. The method of claim 15 further comprising the step of displaying on the wireless receiver the patient, sensor location, room number, patient location, and/or patient number.

19. The method of claim 15 further comprising the step of activating the motion sensor using the wireless receiver.

20. The method of claim 15 further comprising the step of configuring an antenna within the care facility to relay the alert signal between the wireless transmitter and the wireless receiver.

21. A system comprising a medical device for protecting a patient from falling by remotely alerting a caregiver of patient movement before the fall using a motion sensor with an alarm, the system comprising:
a care facility using the motion sensor with the alarm;
a wireless transmitter connected to an operating module integrated into the motion sensor to convert the alarm to a silent alert signal broadcast within the care facility;
a wireless receiver worn by the caregiver for receiving and displaying the alert signal from the wireless transmitter; and
the alert signal to thereby identify the motion sensor that was activated so the caregiver can respond to prevent the patient from falling.
